(19) United States
(12) Patent Application Publication
(10) Pub. No.: US 2022/0153218 A1
(43) Pub. Date: May 19, 2022

(54) CAR ASSISTANCE SYSTEM TO ACTIVATE DISPATCH AND RELAY MEDICAL INFORMATION

(71) Applicant: Panasonic Automotive Systems Company of America, Division of Panasonic Corporation of North America, Peachtree City, GA (US)

(72) Inventor: LaShanna Lynnette Williams, Sterling Heights, MI (US)

(21) Appl. No.: 17/519,930

(22) Filed: Nov. 5, 2021

Related U.S. Application Data
(60) Provisional application No. 63/114,069, filed on Nov. 16, 2020.

(51) Int. Cl. B60R 21/013; B60W 40/08; G16H 10/60; G06K 9/00

(52) U.S. Cl. CPC B60R 21/013; B60W 40/08; G16H 10/60; B60R 2021/0027; G06K 9/00832; G06K 9/00221; G06K 9/00791

(57) ABSTRACT

A communication system for a motor vehicle having a plurality of seats includes a plurality of occupant sensors each detecting the presence of a passenger in a respective one of the seats. Passenger identification means identifies each of the passengers detected by the occupant sensors. A database stores medical information in association with each of the identified passengers. Detecting means detects that the motor vehicle has been in a crash. A transmitter wirelessly communicates with a remote emergency responder. An electronic processor responds to the detecting means detecting that the motor vehicle has been in a crash by causing the transmitter to transmit a signal to the remote emergency responder. The signal informs the remote emergency responder of the respective medical information and seat location associated with each passenger of the motor vehicle.

### CROSS-REFERENCED TO RELATED APPLICATIONS

This application claims benefit of U.S. Provisional Application No. 63/114,069, filed on Nov. 16, 2020, the disclosure of which is hereby incorporated by reference in its entirety for all purposes.

### BACKGROUND OF THE INVENTION

### 1. Field of the Invention

The present invention relates to a communication system, and, more particularly, to a communication system in a motor vehicle.

### 2. Description of the Related Art

When automotive accidents happen, there is not always someone to witness the accident. A person who has been in an accident may need emergency assistance, but may have no way of calling for help themselves.

Even if there is someone there to witness the accident and call for assistance, they will not know any medical history of the occupant to alert emergency respondents. Not knowing a person's medical condition/allergies could lead to unnecessary death or permanent injury.

Some things that can be put in an occupant's profile that would be important to a respondent team to know in order to better assist them would be the occupant's allergies to latex or certain medications. Someone could have autism and a police officer may mistakenly see the victim's behavior as threatening due to lack of knowledge of the condition. Information available to respondent teams such as these and other information could be instrumental in helping to save a person's life.

There are things to bring medical awareness to respondent teams such as bracelets, necklaces and seatbelt covers. However, these are not real time solutions, and do not always stay with the occupants. Seatbelt covers need to be removed each time the occupant leaves the car. If not removed, seatbelt covers may provide false information if someone else is sitting in that seat.

### SUMMARY OF THE INVENTION

The invention may detect a crash and activate a dispatcher service to assist occupants automatically. The invention may also call a respondent team and provide them with data about the occupants' medical information.

The invention may provide a motor vehicle system that alerts a dispatch service that assistance is needed and relays important medical information of the passengers automatically and without the passengers' intervention. The passenger may manually log their medical information through a computer, an app, or the car system itself. That information is shared locally, or in the cloud, in a database. The system can identify occupants via a brought-in-device (e.g., a cell phone) and/or facial recognition.

The motor vehicle may detect a passenger through a seat sensor, seatbelt sensor, floor sensor, and/or a sensor to detect a brought-in-device. The seat sensor may detect weight on the seat. The seatbelt sensor may detect that someone is using the seatbelt. The floor sensor may detect weight on the floor. Once the car detects a passenger, the displays in the vehicle (e.g., front dash display, back seat displays) may become interactive and either ask the passenger through face recognition to confirm who they are or ask the passenger to input their identifying information. The passenger may also be identified by the vehicle communicating with the passenger's brought-in device.

If the vehicle detects a crash (for example, via airbag inflation, a sudden drop in speed, or loss of critical function etc.), a dispatch service is notified automatically. Passengers have the option to manually activate the dispatch service as well.

Once the dispatch services are notified, they can try to speak with the passengers to learn of the situation. If there is no response, the dispatcher may call a respondent team to the scene by telling them the location of the car. The dispatcher may also pull the medical file and communicate the medical information of the occupants to the respondent team. The respondent team may arrive already knowing what they are going into and are better equipped to service the occupants based on their medical information in the database.

The invention may get faster responses to passengers who need assistance. The invention may also help medical responders to better assist passengers with medical disabilities and/or allergies. This invention can help save lives of passengers who may venture off the road for whatever reason when there is no one around to help. The invention may save the lives of accident victims by enabling a faster response when the accident victims are in need and by letting the response team know what to expect when they arrive at the accident scene.

In one embodiment, occupant detection may be done with pressure sensors or seatbelt sensors. The driver may manually set and identify the active occupants instead of it being done automatically.

The system can be used not only in cars but also on school busses, airplanes, semi-trucks, trains and any other public transportation. The medical database may be used in the school system itself for children with any medical issues or allergies, and in companies for their employees. The database may also be used in homes.

The invention comprises, in one form thereof, a communication system for a motor vehicle having a plurality of seats, including a plurality of occupant sensors each detecting the presence of a passenger in a respective one of the seats. Passenger identification means identifies each of the passengers detected by the occupant sensors. A database stores medical information in association with each of the identified passengers. Detecting means detects that the motor vehicle has been in a crash. A transmitter wireles sly communicates with a remote emergency responder. An electronic processor is communicatively coupled to the occupant sensors, the passenger identification means, the database, the detecting means, and the transceiver. The electronic processor responds to the detecting means detecting that the motor vehicle has been in a crash by causing the transmitter to transmit a signal to the remote emergency responder. The signal informs the remote emergency responder of the respective medical information and seat location associated with each passenger of the motor vehicle.

The invention comprises, in another form thereof, a communication method for a motor vehicle having a plurality of seats, including detecting, for each seat, whether there is a passenger in the seat. Each of the detected passengers are identified. Medical information is stored in a database in association with each of the identified passengers. It is ascertained that the motor vehicle has been in a crash. In response to the ascertaining that the motor vehicle has been in a crash, a signal is transmitted to a remote emergency responder. The signal informs the remote emergency responder of the respective medical information and a seat location associated with each passenger of the motor vehicle.

The invention comprises, in yet another form thereof, a medical information communication system including a motor vehicle and a remote database. The motor vehicle includes a plurality of seats, and passenger identification means for identifying each respective passenger in each of the seats. Detecting means is for detecting that the motor vehicle has been in a crash. A transmitter wirelessly communicates with a remote emergency responder. An electronic processor is communicatively coupled to the passenger identification means, the detecting means, and the transceiver. The electronic processor responds to the detecting means detecting that the motor vehicle has been in a crash by causing the transmitter to transmit a signal to the remote emergency responder. The signal informs the remote emergency responder of the identity and a seat location associated with each passenger of the motor vehicle. The database is disposed remotely from the motor vehicle. The database stores medical information in association with each of the identified passengers. The medical information in the database is accessed by the emergency responder.

An advantage of the invention is that it provides a medical database that includes medical information about all passengers in the vehicle including the driver, adults, children and pets. The invention also enables responders to know how many people are in the car and if they need to look further outside the vehicle to identify other passengers that were supposed to be in the car.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a block diagram of one embodiment of a vehicle assistance arrangement of the present invention.

FIG. 2 is a flow chart of one embodiment of a vehicle assistance method of the present invention.

FIGS. 3-5 are database examples of user interface monitor screens and profile information.

FIG. 6 is a flow chart of one embodiment of a communication method of the present invention for a motor vehicle having a plurality of seats.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

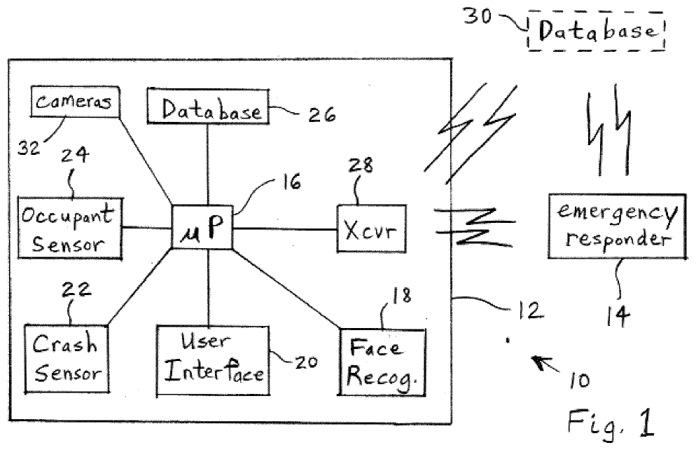

FIG. 1 illustrates one embodiment of a vehicle assistance arrangement 10 of the present invention, including a motor vehicle 12 in wireless communication with an emergency responder 14. Motor vehicle 12 includes an electronic processor 16 in communication with each of a face recognition module 18, a user interface 20, a crash sensor 22, an occupant sensor 24, a database 26, and a transceiver 28. Transceiver 28 may include a transmitter and a receiver.

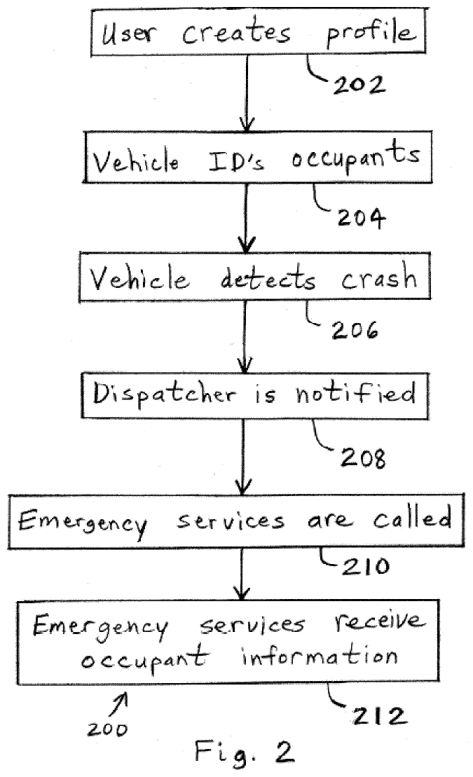

FIG. 2 illustrates one embodiment of a vehicle assistance method 200 of the present invention in which a medical database is relayed to a respondent team. In a first step 202, a profile is created for each user occupant of a motor vehicle 12. Each profile may be entered via a computer, mobile app, or user interface 20, which may include a touch-sensitive display screen, pushbuttons and microphone. The profile may list all relevant medical conditions of each occupant, such as allergies and diseases of the occupant.

Next, in step 204, the vehicle 12 identifies its occupants. Processor 16 may determine that there are occupants in vehicle 12 by use of occupant sensor 24, such as a vehicle occupant camera, a seat sensor, a seatbelt sensor, a floor sensor, etc. Processor 16 may identify the occupants through manual input into user interface 20 by one or more occupants; by communicating (e.g., Bluetooth) with the occupant's brought-in device (e.g., cell phone); and/or by use of facial recognition module 18, for example.

In a next step 206, vehicle 12 detects a crash. For example, the crash may be sensed by crash sensor 22, through air bag deployment, by a sudden drop in speed, by the loss of a critical vehicle function, etc.

In step 208, a dispatcher of emergency services is notified. For example, processor 16 may automatically cause transceiver 28 to send a wireless (e.g., cellular) signal to emergency responder 14. The signal may include the occupants' medical information profiles as retrieved from database 26. Alternatively, the signal may include the occupants' identities and seat positions, and responder 14 may retrieve their medical information profiles from an optional remote database 30. An occupant can also respond manually and speak with the dispatcher if the occupant is able to.

Next, in step 210, emergency services are called. The dispatcher calls emergency services with the occupants' medical information from the profiles.

In a final step 212, emergency services receive the occupant information. Thus, emergency services are able to assist the occupants efficiently.

Crash sensor 22 detects that the vehicle has crashed and informs processor 16. Processor 16 and transceiver 28 may be in communication with dispatch service 14 for assistance and GPS (not shown) for location purposes. When the vehicle crashes, the crash sensor 22 is activated and dispatch service 14 is alerted. Alternatively, a passenger can activate the dispatch service 14 manually. Once dispatch service 14 is activated, the passenger can respond verbally and speak to the dispatcher via a microphone of user interface 20 to ask for assistance or to reject assistance. If no response is given, the dispatch service 14 may view database 26 or database 30 to see which passengers are in the vehicle and their seat locations and then send for a rescue service, informing the rescue service of the passengers and any pertinent information the rescue service may need to know about the passengers. Dispatch service 14 can also notify an emergency contact to let the emergency contact know the situation and which hospital rescue services are taking the passengers to. Once rescue services arrive, the passengers can manually disconnect dispatch service 14. Alternatively, dispatch service 14 can disconnect themselves once they know the rescue services have arrived and have taken over.

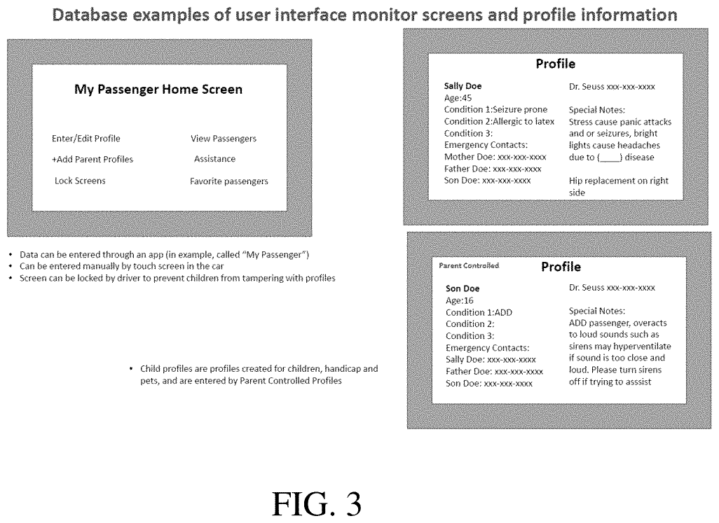

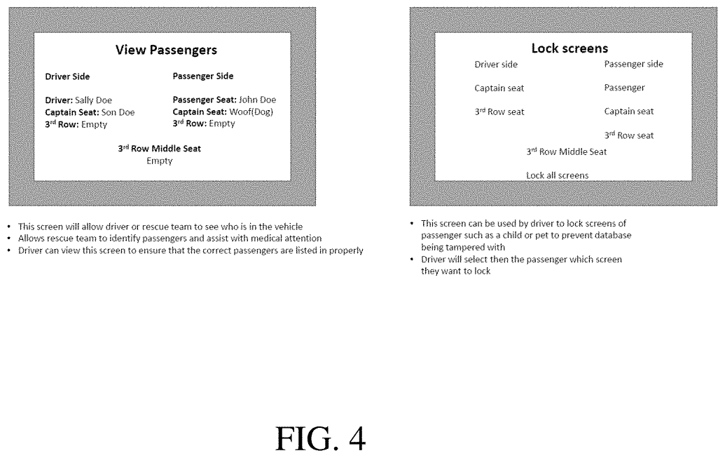

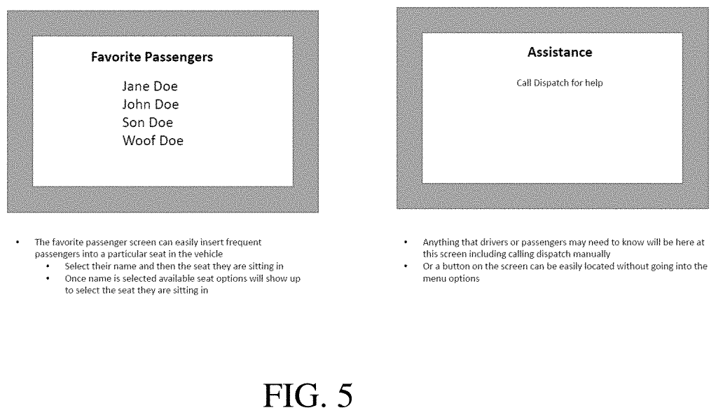

FIGS. 3-5 are database examples of user interface monitor screens and profile information.

In another embodiment, processor 16 (FIG. 1) is connected to and in communication with car cameras 32 that may be attached to vehicle 12 and may be used for traffic monitoring. Cameras 32 may capture images of scenes in front of, behind, and/or on either side of vehicle 12 before, during, and after an accident. Once the dispatcher is called, after numerous attempts by the dispatcher to communicate with the occupants and there is no response, the dispatcher can, through processor 16, access the images captured by cameras 32 in order to get a better view of the current situation and/or the situation before and/or during the crash. This feature may be particularly beneficial to the dispatcher and/or to police authorities in the event that a crime has been committed or is being committed and is related to or involved with the crash.

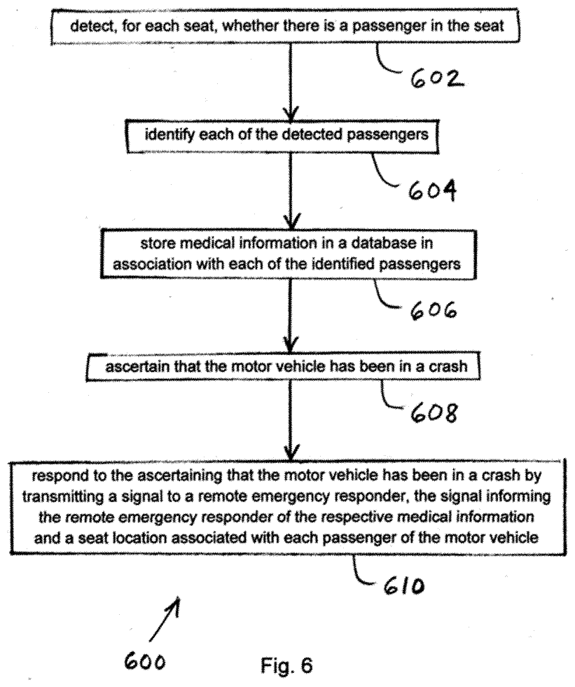

FIG. 6 illustrates one embodiment of a communication method 600 of the present invention for a motor vehicle having a plurality of seats. In a first step 602, it is detected, for each seat, whether there is a passenger in the seat. For example, processor 16 may detect whether there are occupants in vehicle 12 by use of occupant sensor 24, such as a vehicle occupant camera, a seat sensor, a seatbelt sensor, a floor sensor, etc.

In a next step 604, each of the detected passengers are identified. For example, processor 16 may identify the occupants through manual input into user interface 20 by one or more occupants; by communicating (e.g., Bluetooth) with the occupant's brought-in device (e.g., cell phone); and/or by use of facial recognition module 18, for example.

Next, in step 606, medical information is stored in a database in association with each of the identified passengers. For example, a profile is created for each identified passenger of motor vehicle 12. Each profile may be entered via a computer, mobile app, or user interface 20, which may include a touch-sensitive display screen, pushbuttons and microphone. The profile may list all relevant medical conditions of each identified passenger, such as allergies and diseases of the occupant. Each identified passenger's profile may be stored in database 26 and/or database 30 in association with the passenger's identity.

In step 608, it is ascertained that the motor vehicle has been in a crash. For example, a crash may be sensed by crash sensor 22, through air bag deployment, by a sudden drop in speed, by the loss of a critical vehicle function, etc.

In a final step 610, the ascertaining that the motor vehicle has been in a crash is responded to by transmitting a signal to a remote emergency responder. The signal informs the remote emergency responder of the respective medical information and a seat location associated with each passenger of the motor vehicle. For example, in response to crash sensor 22 detecting a crash, processor 16 may automatically cause transceiver 28 to send a wireless (e.g., cellular) signal to emergency responder 14. The signal may include the occupants' medical information profiles as retrieved from database 26 as well as the occupants' identities and seat positions.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A communication system for a motor vehicle having a plurality of seats, the system comprising:
a plurality of occupant sensors each configured to detect the presence of a passenger in a respective one of the seats;
passenger identification means for identifying each of the passengers detected by the occupant sensors;
a database storing medical information in association with each of the identified passengers;
means for detecting that the motor vehicle has been in a crash;
a transmitter configured to wirelessly communicate with a remote emergency responder; and
an electronic processor communicatively coupled to the occupant sensors, the passenger identification means, the database, the detecting means, and the transceiver, the electronic processor being configured to respond to the detecting means detecting that the motor vehicle has been in a crash by causing the transmitter to transmit a signal to the remote emergency responder, the signal informing the remote emergency responder of the respective medical information and a seat location associated with each said passenger of the motor vehicle.

2. The system of claim 1 wherein the signal informs the remote emergency responder of the respective identity of each said passenger of the motor vehicle.

3. The system of claim 1 wherein the occupant sensors include at least one seat sensor, seatbelt sensor, floor sensor, and/or a sensor to detect a brought-in-device.

4. The system of claim 1 wherein the passenger identification means includes a facial recognition module, a sensor to detect a brought-in-device, and/or a user interface configured to receive identity information entered by a passenger.

5. The system of claim 1 wherein the database is disposed in the vehicle.

6. The system of claim 1 wherein the database is disposed at a location remote from the vehicle.

7. The system of claim 1 wherein the detecting means comprises a crash sensor, an airbag inflation sensor, a speed sensor, or a sensor that detects a loss of a critical function, the system further comprising at least one camera configured to capture images of a scene outside of the vehicle and transmit the captured images to the remote emergency responder.

8. A communication method for a motor vehicle having a plurality of seats, the method comprising:
detecting, for each said seat, whether there is a passenger in the seat;
identifying each of the detected passengers;
storing medical information in a database in association with each of the identified passengers;
ascertaining that the motor vehicle has been in a crash; and
responding to the ascertaining that the motor vehicle has been in a crash by transmitting a signal to a remote emergency responder, the signal informing the remote emergency responder of the respective medical information and a seat location associated with each said passenger of the motor vehicle.

9. The method of claim 8 wherein the signal informs the remote emergency responder of the respective identity of each said passenger of the motor vehicle.

10. The method of claim 8 wherein the detecting step is performed by using at least one seat sensor, seatbelt sensor, floor sensor, and/or a sensor for detecting a brought-in-device.

11. The method of claim 8 wherein the identifying is performed by using a facial recognition module, a sensor to detect a brought-in-device, and/or a user interface that receives identity information entered by a passenger.

12. The method of claim 8 wherein the database is disposed in the vehicle.

13. The method of claim 8 wherein the database is disposed at a location remote from the vehicle.

14. The method of claim 8 wherein the ascertaining is performed using a crash sensor, an airbag inflation sensor, a speed sensor, or a sensor that detects a loss of a critical function.

15. A medical information communication system, comprising:
a motor vehicle including:
a plurality of seats;
passenger identification means for identifying each respective passenger in each of the seats;
means for detecting that the motor vehicle has been in a crash;
a transmitter configured to wirelessly communicate with a remote emergency responder; and
an electronic processor communicatively coupled to the passenger identification means, the detecting means, and the transceiver, the electronic processor being configured to respond to the detecting means detecting that the motor vehicle has been in a crash by causing the transmitter to transmit a signal to the remote emergency responder, the signal informing the remote emergency responder of the identity and a seat location associated with each said passenger of the motor vehicle; and
a database disposed remotely from the motor vehicle, the database being configured to store medical information in association with each of the identified passengers and to be accessed by the emergency responder.

16. The system of claim 15 wherein the passenger identification means includes a facial recognition module, a sensor to detect a brought-in-device, and/or a user interface configured to receive identity information entered by a passenger.

17. The system of claim 15 wherein the detecting means comprises a crash sensor, an airbag inflation sensor, a speed sensor, or a sensor that detects a loss of a critical function.

18. The system of claim 15 wherein the motor vehicle includes a plurality of occupant sensors each configured to detect the presence of a passenger in a respective one of the seats.

19. The system of claim 18 wherein the occupant sensors include at least one seat sensor, seatbelt sensor, floor sensor, and/or a sensor to detect a brought-in-device.

20. The system of claim 15 wherein the database is communicatively coupled to the motor vehicle.
